(19) United States
(12) Patent Application Publication
(10) Pub. No.: US 2016/0310406 A1
(43) Pub. Date: Oct. 27, 2016

(54) DISPOSABLE BODY CLEANSING TOWELETTE

(71) Applicant: Peter Marcus Paradigm, LLC, Winterpark, FL (US)

(72) Inventors: Samuel Marcus Nebel, Atlanta, GA (US); Peter Charles Siciak, Atlanta, GA (US)

(21) Appl. No.: 15/139,913

(22) Filed: Apr. 27, 2016

Related U.S. Application Data
(60) Provisional application No. 62/153,018, filed on Apr. 27, 2015.

(51) Int. Cl. A61K 8/97; A01N 65/18; A01N 65/42; A01N 61/00; A61K 8/02; A61Q 19/10; A01N 65/22; A61K 8/92; A01N 25/34; A01N 65/28; A01N 65/08

(52) U.S. Cl. CPC A61K 8/97; A01N 65/28; A01N 65/18; A01N 65/42; A01N 61/00; A01N 65/08; A61Q 19/10; A01N 65/22; A61K 8/925; A01N 25/34; A61K 8/0208; A61K 2800/28

(57) ABSTRACT

Disclosed are formulations and compositions comprising the formulations made up of at least tea tree oil, peppermint oil, castor oil, aloe, and lanolin. Also disclosed are cleansing wipes comprising a substrate and a cleansing formulation, wherein said cleansing formulation comprises tea tree oil; peppermint oil; castor oil; aloe; and lanolin. Disclosed are cleansing products comprising a sealed package and at least one cleansing wipe. Also disclosed are methods of cleansing skin, refreshing/rejuvenating the user, cooling or resetting the body temperature and exfoliating, smoothing, and softening of the skin.

### CROSS-REFERENCE TO RELATED APPLICATIONS

This application claims benefit of U.S. Provisional Application No. 62/153,018 filed Apr. 27, 2015, and is hereby incorporated herein by reference in its entirety.

### BACKGROUND

Perspiration is normal throughout the day, even when an individual is not undergoing or performing any strenuous activity. For instance, a leisurely walk may result in increased body heat, which is followed by the body's natural cooling response—perspiration.

The perspiration may lead to discomfort, including, for example, the radiation of human odors or an accumulation of moisture across various body parts of the individual. The moisture, in turn, may be absorbed by the clothing worn by the individual. Clothing may retain residue of the perspiration, including mild stains and odors.

These consequences are natural but particularly unpleasant when, for example, the individual must traverse through a humid environment en route to a social engagement (e.g., a meeting, a formal event, etc.). The individual may need to freshen up or clean off, but may not have the access or time to take a bath or shower. Moreover, an accessible bathing area may not have any materials to efficiently provide a fresh or clean feeling after the perspiration has occurred.

Further still, in some instances, the individual may desire to cleanse their body even if no perspiration has occurred. Accordingly, there exists a need for a portable means to provide a cleansed and refreshed feeling for an individual, no matter where or when the individual may need or desire to tend to their personal hygiene. The invention is directed to these, as well as other, important needs.

### BRIEF SUMMARY

Disclosed are formulations comprising tea tree oil, peppermint oil, castor oil, aloe and lanolin.

Disclosed are formulations comprising about 0.05% by weight to about 0.3% by weight, based on the total weight of the formulation, of tea tree oil; about 0.05% by weight to about 0.25% by weight, based on the total weight of the formulation, of peppermint oil; about 0.05% by weight to about 1.0% by weight, based on the total weight of the formulation, of castor oil; about 0.05% by weight to about 2.0% by weight, based on the total weight of the formulation, of aloe; and about 0.05% by weight to about 0.1% by weight, based on the total weight of the formulation, of lanolin.

Disclosed are compositions comprising formulations comprising tea tree oil, peppermint oil, castor oil, aloe and lanolin.

Disclosed are cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material.

Disclosed are cleansing products comprising a sealed package; and at least one cleansing wipe comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material.

Disclosed are methods of cleansing skin, comprising contacting the disclosed cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material with said skin to a time sufficient to clean at least a portion of said skin.

Disclosed are methods of exfoliating skin, comprising the disclosed cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material with said skin to a time sufficient to exfoliate at least a portion of said skin.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. Definitions

It must be noted that as used herein and in the appended claims, the singular forms “a”, “an”, and “the” include plural reference unless the context clearly dictates otherwise. Thus, for example, reference to “a cleansing wipe” includes a plurality of such cleansing wipes, reference to “the cleansing wipe” is a reference to one or more cleansing wipe and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

“Biodegradable” refers to the ability of something to be decomposed by bacteria or other biological means. For example, a cleansing wipe that is biodegradable can be a substrate that can be decomposed by a biological means. Decomposition can occur in the soil or in standard waste removal processes.

“Disposable” refers to a material that is intended to be thrown away after use. It can be a single use or multiple uses but the end result is being thrown away. For example, a disposable cleansing wipe is a cleansing wipe that can be used one time and is then thrown away.

“Compostable” refers to the ability of something to be broken down or decomposed into nutrients for the soil.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

B. Formulations

Disclosed are formulations comprising tea tree oil, peppermint oil, castor oil, aloe and lanolin.

Disclosed are formulations comprising about 0.05% by weight to about 0.3% by weight, based on the total weight of the formulation, of tea tree oil; about 0.05% by weight to about 0.25% by weight, based on the total weight of the formulation, of peppermint oil; about 0.05% by weight to about 1.0% by weight, based on the total weight of the formulation, of castor oil; about 0.05% by weight to about 2.0% by weight, based on the total weight of the formulation, of aloe; and about 0.05% by weight to about 0.1% by weight, based on the total weight of the formulation, of lanolin.

In some instances, the formulations can further comprise ginseng. For example, the ginseng can be, but is not limited to being, present in about 0.05% by weight to about 0.3% by weight, based on the total weight of the formulation.

In some instances, the formulation can further comprise an antimicrobial material selected from the group selected from an antibacterial agent, an antiviral agent, an antifungal agent, and combinations thereof. In some instances, the antifungal kills or inhibits the growth of mold, mildew, algae, yeast, spores, or a combination thereof.

In some instances, the formulation can further comprise fragrance. The fragrances can be, but are not limited to, for example, cedar, almond, lemongrass, rose, lavender, blueberry, strawberry, chocolate, and many others. The fragrance can be a natural or artificial fragrance.

In some instances, the formulation can comprise the following:

Polyoxyethylene Hydrogenated Castor Oil

Propylene Glycol

Glycerinum

IPB C-II

MIT

Mentha Piperita Oil

PPG-40-PEG-60 Lanolin Oil

Aloe Barbadensis Extract

Deionized (DI) Water

Sodium Laureth sulfate

Methyl Ionone

Linalool

Tetrasodium EDT

Sodium Citrate

Disodium cocoamphodiacetate

Ginseng

Tea Tree Oil

In some instances, the formulation can comprise the following based on the percent by weight of the formulation.

Polyoxyethylene Hydrogenated Castor Oil 0.6%

Propylene Glycol 3%

Glycerinum 2%

IPBC-II 0.05%

MIT 0.08%

Mentha Piperita Oil 0.3%

PPG-40-PEG-60 Lanolin Oil 0.1%

Aloe Barbadensis Extract 2%

Deionized (DI) Water

Sodium Laureth sulfate 0.1%

Methyl Ionone 0.1%

Linalool 0.1%

Tetrasodium EDT 0.01%

Sodium Citrate 0.1%

Disodium cocoamphodiacetate 0.1%

Ginseng 0.15%

Tea Tree Oil 0.1%

Different formulations can be used based on climate (i.e. temperature), skin sensitivity, portion of skin being targeted, or sex of subject applying the formulation to their skin. For example, the formulation can be customized based on the climate. Different formulations can be used in hot, cold, humid or dry conditions. For example, the formulation can be customized for males and females in a similar manner as deodorants.

In some instances, the disclosed formulations can further comprise one or more of the following:

menthol

alcohol

coconut oil

lemongrass oil

theive's oil

cranberry fruit extract

cucumber fruit extract

panthenol

pineapple fruit extract

argan oil

shea butter

sunflower seed oil

apricot kernel oil

jojoba seed oil

benzalkonium chloride

Royal Jelly

ethanol

soap

vitamin e

oat extract

lemon extract

essential oils

coffee bean extract

green tea extract

oolong extract

vitamin d

vanilla bean extract

cacao bean extract

rosemary

eucalyptus

soy bean oil

squalene

papaya extract

honey

geranium

clary sage

sunflower seed extract

rosewater

ajowan seed

antiperspirant

In some instances, the disclosed formulations can further comprise the following ranges (based on the percent by weight of the formulation).

menthol 0-0.3%

alcohol 0-0.3%

coconut oil 0-0.3%

lemongrass oil 0-0.3%

theive's oil 0-0.3%

cranberry fruit extract 0-0.2%

cucumber fruit extract 0-0.2%

panthenol 0-0.02%

pineapple fruit extract 0-0.2%

argan oil 0-0.3%

shea butter 0-0.1%

sunflower seed oil 0-0.3%

apricot kernel oil 0-0.3%

jojoba seed oil 0-0.3%

benzalkonium chloride 0-0.3%

Royal Jelly 0-0.3%

ethanol 0-0.3%

soap 0-0.3%

vitamin e 0-0.3%

oat extract −0-0.3%

lemon extract 0-0.3%

any essential oils→essential oils 0-0.3%

coffee bean extract 0-0.3%

green tea extract 0-0.3%

oolong extract 0-0.3%

vitamin d 0-0.3%

vanilla bean extract 0-0.3%

cacao bean extract 0-0.3%

rosemary 0-0.3%

eucalyptus 0-0.3%

soy bean oil—0-0.3%

squalene—0-0.3%

papaya extract 0-0.3%

honey 0-0.3%

geranium 0-0.3%

clary sage 0-0.3%

sunflower seed extract 0-0.3%

rosewater 0-0.3%

ajowan seed 0-0.3%

antiperspirant—0-0.3%

In some instances, the bottom range for the disclosed materials can be 0.05%.

In some instances, the disclosed formulations can further comprise a mosquito repellant, a deodorant, an antiperspirant, make up removers, or combinations thereof.

In some instances, the disclosed formulations, when applied to a surface such as, but not limited to, skin, do not dry out the surface or leave a film or residue or stickiness on the surface. Thus, the disclosed formulations, when applied to a surface, can leave a clean, fresh feeling.

The disclosed formulations can be applied to a surface by way of a substrate comprising the formulation. In some instances, the disclosed formulations can be added to a substrate and then wiped onto a surface. In some instances, the disclosed formulations can be applied directly to a surface by pouring or spraying on the surface.

### C. Products

Disclosed are products comprising the formulations disclosed herein and a substrate. In some instances, the substrate can be impregnated with one of the disclosed formulations.

In some instances a substrate can be comprised of, but is not limited to, polyester, wood-pulp, bamboo, cellulose-based fibers, cotton, rayon, hemp, wool, spandex, polyolefins, polyamide like nylon, aramids, poly, acrylic, or similar microfibers or nanofibers and other known materials. In some instances, a substrate can be, for example, 50% polyester and 50% viscose (wood-pulp) or 100% viscose.

In some instances, the substrate can be biodegradable, disposable, compostable, or a combination thereof. In some instances, biodegradable can refer to a substrate that can fully disintegrate in the soil in days, weeks, or months. In some instances, a substrate can fully disintegrate in the soil in six weeks.

In some instances, the substrate can be created using methods such as, for example, methods to create hydrospun, spunlaced (hydroentanglement), needle-punched, flashspun, melt-blown, bonded by heat with a sealer, curing, calendaring, ultrasonic pattern bonding, or chemical bonding air-laid materials. The substrate can further be embossed to provide texture. Texturing can include adding pores or exfoliating beads to the substrate. The texture can be used to, for example, provide a deeper cleanse of the applicant's skin. Texture can provide a bigger surface area as well.

Substrates can be a variety of shapes and sizes. In some instances, substrates can be rectangular, square, or circular in shape. In some instances, substrates can contain any number of sides, for example, substrates can be a pentagon, hexagon, heptagon, octagon, etc. In some instances, substrates can have at least one side that is about 4, 5, 6, 7, 8, 9, 10, 11, 12, 13, 14, 15, 16, 17, 18, 19, 20, 21, 22, 23, or 24 inches. In some instances, a second side is at least the same size as a first side. In some instances, a second side is at least 0.5, 1, 2, 3, 4, or 5 inches longer than a first side. In some instances, substrates can be at least about 6 inches by about 6 inches, about 8 inches by about 10 inches, about 10 inches by about 12 inches, about 12 inches by about 9 inches, about 15 inches by about 12 inches, about 9 inches by 11 inches. Sizes can be determined based on the body part(s) they can be used for. For example, a cleansing wipe for the face or hands can be smaller than a cleansing wipe needed for the arms, legs or torso.

In some instances, substrates can be comprised of two or more layers. Each layer can be composed of the same material or different materials. In some instances, each layer can be soaked in the disclosed formulations individually before combining into the substrate.

In some instances, substrates can range from 45 grams per square meter (gsm) to 85 gsm. In some instances, substrates can be 55, 60, or 65 gsm.

1. Cleansing Wipe

Disclosed are cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material. In some instances, the fragrance can be lavender. In some instances, the fragrance can be, but is not limited to, for example, cedar, almond, lemongrass, rose, lavender, blueberry, strawberry, chocolate, and many others. The fragrance can be a natural or artificial fragrance. In some instances, the antimicrobial material is selected from the group selected from an antibacterial agent, an antiviral agent, an antifungal agent, and combinations thereof. In some instances, the antifungal kills or inhibits the growth of mold, mildew, algae, yeast, spores, or a combination thereof.

The formulation can be any of the formulations disclosed herein. In some instances, cleansing wipes comprise a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said tea tree oil is present at a level of about 0.05% by weight to about 0.3% by weight, based on the total weight of the cleansing formulation.

In some instances, cleansing wipes comprise a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said peppermint oil is present at a level of about 0.05% by weight to about 0.25% by weight, based on the total weight of the cleansing formulation.

In some instances, cleansing wipes comprise a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said castor oil is present at a level of about 0.05% by weight to about 1.0% by weight, based on the total weight of the cleansing formulation.

In some instances, cleansing wipes comprise a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said aloe is present at a level of about 0.05% by weight to about 0.2% by weight, based on the total weight of the cleansing formulation.

In some instances, cleansing wipes comprise a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said lanolin is present at a level of about 0.05% by weight to about 0.1% by weight, based on the total weight of the cleansing formulation.

In some instances, for example, the formulation can comprise about 0.05% by weight to about 0.3% by weight, based on the total weight of the formulation, of tea tree oil; about 0.05% by weight to about 0.25% by weight, based on the total weight of the formulation, of peppermint oil; about 0.05% by weight to about 1.0% by weight, based on the total weight of the formulation, of castor oil; about 0.05% by weight to about 2.0% by weight, based on the total weight of the formulation, of aloe; and about 0.05% by weight to about 0.1% by weight, based on the total weight of the formulation, of lanolin.

Disclosed are cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material and wherein said cleansing formulation further comprises water.

In some instances, the disclosed cleansing wipes can be biodegradable, disposable, compostable, or a combination thereof.

Disclosed are cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said substrate is at least about 12 inches by about 9 inches.

Disclosed are cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said substrate comprises cellulose fiber. In some instances a substrate can be comprised of, but is not limited to, polyester, wood-pulp, bamboo, cellulose-based fibers, cotton, rayon, hemp, wool, spandex, polyolefins, polyamide like nylon, aramids, poly, acrylic, or similar microfibers or nanofibers, and other known materials. In some instances, a substrate can be, for example, 50% polyester and 50% viscose (wood-pulp) or 100% viscose.

In some instances, the substrate can be biodegradable, disposable, or both.

In some instances, the substrate can be created using methods such as, for example, methods to create hydrospun, spunlaced (hydroentanglement), needle-punched, flashspun, melt-blown, bonded by heat with a sealer, curing, calendaring, ultrasonic pattern bonding, or chemical bonding air-laid materials. The substrate can further be embossed to provide texture. Texturing can include adding pores or exfoliating beads to the substrate. The texture can be used to, for example, provide a deeper cleanse of the applicant's skin. Texture can provide a bigger surface area as well.

Substrates can be a variety of shapes and sizes. In some instances, substrates can be rectangular, square, or circular in shape. In some instances, substrates can contain any number of sides, for example, substrates can be a pentagon, hexagon, heptagon, octagon, etc. In some instances, substrates can have at least one side that is about 6, 7, 8, 9, 10, 11, 12, 13, 14, 15, 16, 17, 18, 19, 20, 21, 22, 23, or 24 inches. In some instances, a second side is at least the same size as a first side. In some instances, a second side is at least 0.5, 1, 2, 3, 4, or 5 inches longer than a first side. In some instances, substrates can be at least about 6 inches by about 6 inches, about 8 inches by about 10 inches, about 10 inches by about 12 inches, about 12 inches by about 9 inches, about 15 inches by about 12 inches, about 9 inches by 11 inches. Sizes can be determined based on the body part(s) they can be used for. For example, a cleansing wipe for the face or hands can be smaller than a cleansing wipe needed for the arms, legs or torso.

In some instances, substrates can be comprised of two or more layers. Each layer can be composed of the same material or different materials. In some instances, each layer can be soaked in the disclosed formulations individually before combining into the substrate.

Disclosed are cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein at least a portion of said substrate is textured.

The term wipe and towelette can be used interchangeably.

2. Cleansing Products

Disclosed are cleansing products comprising a sealed package; and at least one of the disclosed cleansing wipes. Thus, disclosed are cleansing products comprising a sealed package; and at least one cleansing wipe comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material.

Disclosed are cleansing products comprising a sealed package; and at least one cleansing wipe comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein said sealed package can be resealable. Sealed packages can be made of any number of materials, such as, but not limited to, plastic, foil, metal, and rubber. In some instances, sealed packages can be a canister. In some instances, sealed packages can be any package that allows for moisture retention and/or prevents contamination. Sealed packages can be resealable or one time use packages. One time use packages can be sealed using any known sealing technique, such as, but not limited to, heat seal, adhesive, hook and loop closures (such as those sold be Velcro), interlocking closure, and zipper. In some instances, resealable can be any method known to be resealable. For example, a resealable package can be resealed using any known resealable technique, such as, but not limited to, a zipper, interlocking closure, snaps, hook and loop closures (such as those sold be Velcro), adhesive, clasps, and a material that ties or tightens. A resealable package can have a lid that opens and closes.

Disclosed are cleansing products comprising a sealed package; and at least one cleansing wipe comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material, wherein at least two cleansing wipes are present. In some instances, up to 5, 10, 15, 20, 25, 30, 35, 40, 45, 50, 55, 60, 65, 70, 75, 80, 85, 90, 95, 100, 150, or 200 cleansing wipes can be present. In some instances, the cleansing wipes are removably joined. Cleansing wipes that are removably joined are those that are connected to each other in a way that can quickly and easily be. For example, removably joined can include, but is not limited to, perforation which allows for the removably joined cleansing wipes to be easily torn apart.

Cleansing products can comprise a dry or wet substrate. Wet substrates comprise the formulation present in the disclosed cleansing wipes. In some instances, cleansing wipes comprise a dry substrate that can be wetted by adding water to the substrate. For example, a dry substrate can comprise the formulation present in the cleansing wipe dried onto or within the substrate.

In some instances, the cleansing product can be stationary. For example, stationary cleansing products can be mounted in restrooms, showers, or any public location.

In some instances, the cleansing product can be portable.

In some instances the cleansing product can comprise more than one sealed package. One sealed package can comprise a cleansing wipe for the upper body and one sealed package can comprise a cleansing wipe for the lower body.

In some instances, the cleansing product can further comprise one or more other wipes such as, but not limited to, other body wipes of different or same scent (same material, biodegradable), flushable wipes for the perineal or perianal area (including hemorrhoid soothing), sunscreen wipes, mosquito repellant wipes, hand wipes, deodorant wipes or antiperspirant wipes specifically for underarms, face wipes, make up removal wipes, and screen wipes for electronics.

### D. Methods of Use

Disclosed are methods of cleansing skin, comprising contacting the disclosed cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material with said skin to a time sufficient to clean at least a portion of said skin. Cleansing skin refers to the removal of debris, dirt, oil, bacteria, sweat, odors, and the like from skin.

In some instances, the antimicrobial material is active (e.g. can kill and/or protect) for at least 24-48 hours upon administration to the skin. In some instances, the antifungal kills or inhibits the growth of mold, mildew, algae, yeast, spores, or a combination thereof.

In some instances, the entire body can be wiped with one cleansing wipe. In some instances, more than one cleansing wipe can be used to cleanse the skin of the entire body.

Disclosed are methods of exfoliating skin, comprising the disclosed cleansing wipes comprising a substrate; and a cleansing formulation; wherein said cleansing formulation comprises: tea tree oil; peppermint oil; castor oil; aloe; lanolin; optionally, ginseng; optionally, fragrance; and optionally, an antimicrobial material with said skin to a time sufficient to exfoliate at least a portion of said skin. Exfoliating skin refers to the removal of dead skin cells from the skin's outmost surface.

In some instances, the antimicrobial material is active (e.g. can kill and/or protect) for at least 24-48 hours upon administration to the skin. In some instances, the antifungal kills or inhibits the growth of mold, mildew, algae, yeast, spores, or a combination thereof.

In some instances, the entire body can be exfoliated with one cleansing wipe. In some instances, more than one cleansing wipe can be used to exfoliate the skin of the entire body.

In some instances, the disclosed formulations, when applied to the skin through use of the disclosed cleansing wipes, can provide a cooling sensation to the skin, with a perceived reduction of body temperature. This can be achieved, for example, but is not limited to, by the presence of peppermint oil (e.g. mentha piperita oil) in the disclosed formulations. In some instances, the body temperature can be cooled or reset.

In some instances, the disclosed formulations can deodorize and/or neutralize human body odor. This can be achieved, for example, but is not limited to, by the presence of tea tree oil, peppermint oil, fragrance, essential oils, or a combination thereof in the disclosed formulations.

In some instances, the disclosed formulations can provide soothing and relaxing aromas and scents absorbed via the nasal passage upon application. This can be achieved by, but is not limited to, the presence of tea tree oil and/or peppermint oil in the disclosed formulations. Further still, in some instances, the disclosed formulations can provide a natural antiseptic. This can be achieved by, for example, applying tea tree oil to the substrate as disclosed herein.

In some instances, the disclosed formulations can be configured to sooth the skin. This can be achieved by, for example, the presence of lanolin oil in the disclosed formulations. Further still, in some instances, the disclosed formulations can stimulate the nervous system by way of the skin, and benefit the immune system. This can be achieved by, for example, at least in part, the presence of ginseng in the disclosed formulations.

In some instances, the disclosed formulations can moisturize and condition the applicant's skin. This can be achieved by, for example, the presence of aloe and/or hydrogenated castor oil in the disclosed formulations. In some instances, the disclosed formulations can smooth or soften the skin.

In some instances, the disclosed formulations can break down, for example, dirt, sweat, grime, odors, bacteria, oils, found on the applicant's skin. This can be achieved by the presence of tea tree oil and/or peppermint oil in the disclosed formulations.

In some instances, the disclosed formulations can act as an insect repellant. This can be achieved by the presence of tea tree oil in the disclosed formulations. Tea tree oil is a natural insect repellant.

In some instances, the disclosed formulations can be applied to clothes, animals, and other surfaces. The formulations can be applied via a substrate comprising the formulation or by directly applying the formulation to the surface such as, but not limited to, pouring or spraying. The application of the disclosed formulations can freshen and/or deodorize clothes. The application of the disclosed formulations can clean or deodorize animals. In some instances, the disclosed formulations can further comprise an anti-flea/tick medication. In some instances, the disclosed formulations can be applied to any surface to remove the smell of smoke.

In some instances, the disclosed formulations can be used to enhance human scent. For example, the formulations can further comprise cologne or pheromones.

The disclosed methods can be performed by applying the disclosed formulations to a surface of interest. Application can occur by using one or more of the disclosed cleansing wipes.

Those skilled in the art will recognize, or be able to ascertain using no more than routine experimentation, many equivalents to the specific embodiments of the method and compositions described herein. Such equivalents are intended to be encompassed by the following claims.

1. A formulation comprising:
about 0.05% by weight to about 0.3% by weight, based on the total weight of the formulation, of tea tree oil;
about 0.05% by weight to about 0.25% by weight, based on the total weight of the formulation, of peppermint oil;
about 0.05% by weight to about 1.0% by weight, based on the total weight of the formulation, of castor oil;
about 0.05% by weight to about 2.0% by weight, based on the total weight of the formulation, of aloe; and
about 0.05% by weight to about 0.1% by weight, based on the total weight of the formulation, of lanolin.

2. The formulation of claim 1, further comprising:
ginseng.

3. The formulation of claim 2, wherein the ginseng is present about 0.05% by weight to about 0.3% by weight, based on the total weight of the formulation.

4. The formulation of claim 1, further comprising:
an antimicrobial material selected from the group selected from an antibacterial agent, an antiviral agent, an antifungal agent, and combinations thereof.

5. The formulation of claim 1, further comprising:
fragrance.

6. A product comprising the formulation of claim 1 and a substrate.

7. The product of claim 2, wherein the substrate is impregnated with the formulation of claim 1.

8. A cleansing wipe comprising:
a substrate; and
a cleansing formulation;
wherein said cleansing formulation comprises:
tea tree oil;
peppermint oil;
castor oil;
aloe;
lanolin;
optionally, ginseng;
optionally, fragrance; and
optionally, an antimicrobial material.

9. The cleansing wipe of claim 8,
wherein said tea tree oil is present at a level of about 0.05% by weight to about 0.3% by weight, based on the total weight of the cleansing formulation.

10. The cleansing wipe of claim 8,
wherein said peppermint oil is present at a level of about 0.05% by weight to about 0.25% by weight, based on the total weight of the cleansing formulation.

11. The cleansing wipe of claim 8,
wherein said castor oil is present at a level of about 0.05% by weight to about 1.0% by weight, based on the total weight of the cleansing formulation.

12. The cleansing wipe of claim 8,
wherein said aloe is present at a level of about 0.05% by weight to about 0.2% by weight, based on the total weight of the cleansing formulation.

13. The cleansing wipe of claim 8,
wherein said lanolin is present at a level of about 0.05% by weight to about 0.1% by weight, based on the total weight of the cleansing formulation.

14. The cleansing wipe of claim 8,
wherein said antimicrobial material is selected from the group selected from an antibacterial agent, an antiviral agent, an antifungal agent, and combinations thereof.

15. The cleansing wipe of claim 8, wherein said cleansing formulation further comprises water.

16. The cleansing wipe of claim 8,
wherein said cleansing wipe is biodegradable, disposable, or compostable.

17. The cleansing wipe of claim 8,
wherein said substrate is at least about 11 inches by about 9 inches.

18. A cleansing product, comprising:
a sealed package; and
at least one cleansing wipe of claim 8.

19. A method of cleansing skin, comprising:
contacting said cleansing wipe of claim 8 with said skin for a time sufficient to clean at least a portion of said skin.

20. A method of exfoliating skin, comprising:
contacting said cleansing wipe of claim 8 with said skin for a time sufficient to exfoliate at least a portion of said skin.
